(19) United States
(12) Patent Application Publication
(10) Pub. No.: US 2006/0247769 A1
(43) Pub. Date: Nov. 2, 2006

(54) Polycrystalline diamond compact surfaces on facet arthroplasty devices

(75) Inventors: Fred Molz, Collierville, TN (US); Hai H. Trieu, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/116,397

(22) Filed: Apr. 28, 2005

(51) Int. Cl. A61F 2/44; A61F 2/28

(52) U.S. Cl. 623/17.11; 623/23.51

(57) ABSTRACT

At least a portion of an articulating surface of a facet arthroplasty device may comprise a polycrystalline diamond compact. The polycrystalline diamond compact may be useful to increase the wearability and decrease the coefficient of friction of the at least one articulating surface of the facet arthroplasty device. The polycrystalline diamond compact may be utilized with any facet arthroplasty device and may be formed by any appropriate method including, but not limited to, diamond sintering, chemical vapor deposition, physical vapor deposition, and energy beam ablation/deposition.

### FIELD OF THE INVENTION

Embodiments of the invention relate to medical implants and facet arthroplasty devices. More particularly, the invention relates to polycrystalline diamond compacts useful as at least a portion of the articulating surfaces of a facet arthroplasty device.

### BACKGROUND

The primary structures of the spinal column are the vertebrae and intervertebral discs. The vertebrae are bones that form the rigid structure of the spine. Several different bony structures can be identified in the vertebrae, including the solid body forming the anterior section of the vertebrae, foramen, pedicles, the transverse process, inferior and superior articular processes, and the spinous process. Intervertebral discs are located between adjacent vertebrae, situated generally between the bodies of the vertebrae. The intervertebral discs provide a cushion between adjacent vertebral bodies and function as an orthopedic joint to allow movement of the vertebrae.

Besides the intervertebral disc, adjacent vertebrae also are connected through their respective articular processes. Inferior articular processes and superior articular processes of adjacent vertebrae form a zygapophyseal, or facet, joint at the posterior of the vertebrae. The facet joints formed by the articular processes of adjacent vertebrae are synovial joints. Synovial joints are joints that are surrounded by a connective tissue and hyalin cartilage and allow the bones to articulate against each other. Facet joints, like intervertebral discs, carry some of the weight of the spinal column and guide movement between adjacent vertebrae.

The facet joints of the spinal column are subject to degenerative diseases and injury, which may lead to pain and immobilization. Facet joint degeneration also has been implicated in various degenerative spinal pathologies such as degenerative spondylolisthesis, central and lateral stenosis, degenerative scoliosis, and kypho-scoliosis. One method to treat a damaged facet joint is by the implantation of an appropriate facet arthroplasty device. Facet arthroplasty devices may replace all or a portion of the facet joint, for example the articular process, or provide additional articulating surfaces to augment the damaged endogenous articulating surfaces.

Exemplary facet arthroplasty devices are disclosed in U.S. Pat. Nos. 5,571,19, 6,419,703, 6,565,605, 6,579,319, 6,610,091, 6,669,729, 6,811,567, and Re 36,758; and U.S. patent application Ser. Nos. 2002/0065557, 2002/0072800, 2002/0123806, 2003/0004572, 2003/0028250, 2003/0040797, 2003/0171750, 2003/0191532, 2003/0204259, 2004/0006391, 2004/0049272, 2004/0049273, 2004/0049274, 2004/0049275, 2004/0049276, 2004/0049277, 2004/0049278, 2004/0049281, 2004/0111154, 2004/0230304, 2005/0010291, 2005/0015146, 2005/0027361, 2005/0033434, 2005/0043797, 2005/0043799, 2005/0049705, and 2005/0055096, the disclosures of each of which are incorporated herein by reference in their entirety.

Facet implants, like the facet joints they replace or augment, generally are designed to support some of the weight of the spinal column and comprise articulating surfaces that allow movement between adjacent vertebrae.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

What is needed is an improved articulating surface for facet arthroplasty devices that increases the wearability of the devices. A surface that has a sufficiently low coefficient of friction so as to allow efficient articulation by the facet arthroplasty device also is needed. A surface that reduces the probability of particle ejection additionally is needed. Embodiments of the invention solve some or all of these needs, as well as additional needs.

Therefore, in accordance with embodiments of the present invention, there is provided a facet arthroplasty device comprising at least one articulating surface, wherein at least a portion of the at least one articulating surface comprises a polycrystalline diamond compact.

Additionally, there is provided a method for modifying a facet arthroplasty device having at least one articulating surface. The method comprises forming a polycrystalline diamond compact on at least a portion of the at least one articulating surface.

These and other features and advantages of the present invention will be apparent from the description provide herein.

### DETAILED DESCRIPTION OF THE EMBODIMENTS

The following description is intended to convey a thorough understanding of the various embodiments of the invention by providing a number of specific embodiments and details involving articulating surfaces for facet arthroplasty devices. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

Throughout this description, the phrase “polycrystalline diamond compact” (“PDC”) refers to a composite structure of polycrystalline diamond and at least one other material. Layers of polycrystalline diamond on a metal substrate is an exemplary PDC. A PDC additionally may comprise a transitional layer positioned between the at least one other material and the polycrystalline diamond. A PDC may be formed, for example, by forming a layer of polycrystalline diamond on a substrate. The combined polycrystalline diamond layer and the substrate, being a composite structure of polycrystalline diamond and at least one other material, may be referred to as a PDC.

It is a feature of an embodiment of the present invention to provide a facet arthroplasty device for replacement or augmentation of a natural facet joint between adjacent vertebral bodies. The facet arthroplasty device comprises at least one articulating surface, wherein at least a portion of the at least one articulating surface includes a polycrystalline diamond compact. Also, it is a feature of an embodiment of the present invention to provide a method for modifying a facet arthroplasty device having at least one articulating surface. The method comprises forming a polycrystalline diamond compact on at least a portion of the at least one articulating surface of the facet arthroplasty device.

Polycrystalline diamond compact articulating surfaces on facet arthroplasty devices may be desirable because of the increased wear resistance of the PDC, when compared to other surfaces, for example metallic, ceramic, and polymeric surfaces. Diamond is the hardest known substance. Forming a PDC, for example, on articulating surfaces of a facet arthroplasty device may help to make the articulating surfaces more durable. Preferably, the PDC articulating surfaces may be so durable that the expected lifetime of the facet arthroplasty device greatly exceeds that of the patient. This may be desirable in order to reduce the probability of the necessity of prosthesis replacement in the future. Additionally, because the diamond surface is so hard, articulating surfaces comprising a polished PDC may have very low frictional resistance, resulting in a smoother, better articulating surface. The low coefficient of friction makes PDC especially desirable as an articulating surface. Also, the hard PDC may be less prone to ejecting debris or particles thereof into the body, when compared to other articulation surfaces.

Table 1 below compares the hardness values of PDC and several other materials.

TABLE 1

| Material | Hardness (Knoop) |
|---|---|
| PDC | 9000 |
| Cubic boron nitride | 4500 |
| Silicon carbide | 2500 |
| Aluminum oxide | 2000 |
| Tungsten carbide | 2200 |
| Silicon nitride | 14.2 |

As can be seen in Table 1, a PDC articulating surface on a facet arthroplasty device may be much harder than other surface materials. As discussed, a harder articulating surface may have several advantages, including increased durability, increased implant lifetime, lower coefficient of friction when polished, and decreased probability of particle ejection. In a preferred embodiment of the invention, the facet arthroplasty device has at least one articulating surface with a Knoop hardness of greater than about 6000. This is thought to not be possible without the use of a PDC articulating surface.

The polycrystalline diamond compact articulating surfaces provided by embodiments of the invention are applicable to all manner of facet arthroplasty devices, in accordance with the guidelines herein. The following description of facet arthroplasty devices to which the PDC articulating surfaces of embodiments of the invention are applicable is exemplary only and it should be appreciated that the invention is not limited thereto.

In one embodiment, the facet arthroplasty device may comprise a first component having a body with at least one male articulation member attached thereto, and a second component having a body with at least one female articulation member attached thereto. The male and female articulation members may engage one another so that the first and second components are articulately connected. For example, the male and female articulation members may be hinges or ball-and-socket joints. The first component may be connected to the posterior of a vertebra and the second component may be connected to the posterior of an adjacent vertebra. U.S. Pat. No. 6,669,729 and U.S. patent application Ser. No. 2003/0171750 describe facet arthroplasty devices according to this description, and are incorporated herein by reference in their entirety.

In another embodiment, the facet arthroplasty device may comprise superior and inferior components roughly in the shape of a hollow cone or pyramid such that the superior and inferior components may fit over, respectively, the distal tips of superior and inferior articular processes of adjacent vertebrae. The superior and inferior components may provide smooth matching surfaces that protect the surfaces of the natural articular processes. U.S. Reissed Pat. No. 36,758 and U.S. Pat. No. 5,571,191 describe facet arthroplasty devices according to this description, and are incorporated herein by reference in their entirety.

In another embodiment, the facet arthroplasty device may be a replacement of the posterior elements of a natural vertebrae. The prosthesis may comprise a pair of prosthetic mounts, a prosthetic lamina extending from the two prosthetic mounts, a pair of prosthetic superior facets extending from the two prosthetic mounts and the prosthetic lamina, a pair of prosthetic inferior facets extending from the prosthetic lamina, a prosthetic spinous process extending from the prosthetic lamina, and a pair of prosthetic transverse processes extending from the two prosthetic mounts. Two posterior element prostheses may be attached to adjacent vertebrae to provide a new facet joint between the superior and inferior facets of the two adjacent posterior elements prostheses. A posterior element prosthesis may be attached to a vertebra by resecting the vertebra at its natural pedicles in order to remove the natural lamina, the two natural superior facets, the two natural inferior facets, the natural spinous process, and the two natural transverse processes, leaving a pair of pedicle end surfaces. The posterior elements prosthesis then may be attached to the natural pedicles, for example, by placing the prosthetic mounts against the pedicle surfaces and securing them with screws. U.S. Pat. No. 6,419,730 and U.S. patent application Ser. No. 2003/0040797 describe facet arthroplasty devices according to this description, and are incorporated herein by reference in their entirety.

In another embodiment, the facet arthroplasty device may comprise a bone contacting surface that contacts an exterior or resected surface of a vertebra, a surface that articulates with a facet of an adjacent vertebra and is connected to the bone contacting surface, and a fixation element that attaches the bone contacting surface to the vertebra, for example, at one of the vertebra's pedicles. The facet arthroplasty device may be configured so that no portion of the device contacts the posterior arch of the vertebra to which it is attached. This prosthesis may replicate the natural anatomy of a facet so that it may replace at least a portion of the bone of a facet located on the vertebra to which it is attached. U.S. Pat. No. 6,579,319 and U.S. patent application Ser. Nos. 2002/0065557, 2003/0004572, and 2003/0191532 describe facet arthroplasty devices according to this description, and are incorporated herein by reference in their entirety.

In another embodiment, the facet arthroplasty device may comprise a prosthesis for the replacement of at least two facets located on a vertebra. The prosthesis may comprise at least one bone contacting surface that is adapted to be secured to a surface of the vertebra connected to at least two bearing surfaces for articulating with facets of an adjacent vertebra. The prosthesis may be configured so that it is not supported by the lamina of the vertebra to which it is attached. In a preferred embodiment, two bone contacting surfaces may abut the pedicles of the vertebra. A bridge may connect the two bone contacting surfaces, to which two articular surfaces may be attached. U.S. Pat. No. 6,565,605 and U.S. patent application Ser. Nos. 2003/0204259 and 2002/0072800 describe facet arthroplasty devices according to this description, and are incorporated herein by reference in their entirety.

In another embodiment, the facet arthroplasty device may comprise a superior implant and an inferior implant, each of which may have an articulating surface and a fixation surface. In a preferred embodiment, the articulating surfaces may be curved to form male and female mating surfaces that are in articular connection with each other. The superior implant may be configured for placement on the superior articular facet and the inferior implant may be configured for placement on the inferior articular facet. The superior and inferior implants may be fixed to the surface of adjacent vertebrae using, for example, bone cement, pegs, pips, ridges grooves, or screws. Preferably, the inferior implants may be configured to be fixed to a vertebra using a translaminer fixation mechanism, for example a long screw. U.S. patent application Ser. No. 2005/0049705 describes facet arthroplasty devices according to this description, and is incorporated herein by reference in its entirety.

In another embodiment, the facet arthroplasty device may comprise superior and inferior components. The superior component may have a generally conical external surface and an internal cavity adapted to be implanted on a tapered resected portion of the superior articular process of a vertebra. The inferior component may have a cup adapted to receive the conical external surface of the superior component and a base adapted to be implanted at a surgically prepared site on an inferior articular process of an adjacent vertebra. U.S. patent application Ser. No. 2005/0043797 describes facet arthroplasty devices according to this description, and is incorporated herein by reference in its entirety.

In another embodiment, the facet arthroplasty device may comprise a first prosthesis for partially replacing the anterior facet, having a first sliding bearing surface, and a second prosthesis for totally replacing the posterior facet, having a second sliding bearing surface presenting at least a portion of a shape that is similar or identical to the shape of at least a portion of the first sliding bearing surface of the first prosthesis. Therefore, each posterior prosthesis presents a surface for bearing against the bone and a surface for bearing in sliding manner on the surface of the corresponding prosthesis. U.S. patent application Ser. No. 2005/0015146 describes facet arthroplasty devices according to this description, and is incorporated herein by reference in its entirety.

In another embodiment, the facet arthroplasty device may comprise an inferior facet replacement device having a head extending from a rod. The head may be spherical or may assume a more anatomical shape to reduce wear and permit a relatively natural range of articulation at the facet joint. At least a portion of the rod may be tubular and carry internal threads. A facet connector having external threads configured to engage the internal threads of the inferior facet replacement device also may be provided, and may be used to connect the inferior facet replacement device to a vertebra. The inferior facet replacement device may articulate with a superior facet replacement device to create an artificial facet joint. The superior facet replacement device may comprise a head extending from a stem. The stem may be configured to fasten to the inferior vertebra, for example, by threads on its external surface. The superior facet replacement device may be, for example, a pedicle screw or an impacted pedicle post. The head of the superior facet replacement device may be a socket configured to accept the head of the inferior facet replacement device and may be selected from a variety of geometries suitable for engaging the inferior facet replacement device. U.S. patent application Ser. No. 2005/0033434 describes facet arthroplasty devices according to this description, and is incorporated herein by reference in its entirety.

In another embodiment, the facet arthroplasty device may comprise a bar element. The bar element may be secured to a vertebral body by at least one fixation element and may carry at least one inferior facet joint structure element. In a preferred embodiment, two fixation elements (left and right) and two inferior facet joint structure elements (left and right) may be used. The prosthesis thereby readily accommodates a double-sided (i.e., left and right) facet joint replacement. The bar element may be sized and shaped to span the distance between left and right pedicles of a vertebral body. Inferior facet joint structure elements may be fixedly attached to the bar element to provide a fixed, pre-ordained spaced apart relationship between the facet surface elements. The inferior facet joint structure elements may be generally concave or cup-shaped, to thereby articulate with generally convex or ball-shaped superior facet joint structures located on an adjacent vertebra. Alternatively, the inferior facet joint structure elements may be generally convex or ball-shaped, to thereby articulate with generally concave or cup-shaped superior facet joint structures. U.S. Pat. Nos. 6,811,567 and 6,610,091, and U.S. patent application Ser. Nos. 2002/0123806, 2003/0028250, 2004/0006391, 2004/0049272, 2004/0049273, 2004/0049274, 2004/0049275, 2004/0049276, 2004/0049277, 2004/0049278, 2004/0049281, 2004/0111154, 2005/0027361, and 2005/0043799 describe facet arthroplasty devices according to this description, and are incorporated herein by reference in their entirety.

In another embodiment, the facet arthroplasty device may comprise superior and inferior facet joint components. The superior facet joint may comprise a longitudinal body with a superior end and an inferior end, the inferior end forming an inner surface. The superior facet joint may additionally comprise a fastener threaded on its distal end to fasten to bone with a groove on its proximal end adapted to receive the superior end portion of the longitudinal body. Also, the superior facet joint may comprise a set screw received within the proximal groove of the fastener which secures the longitudinal element to the fastener. The inferior facet joint may be analogous to the superior facet joint, except that the inferior end portion of the longitudinal body may form an outer surface. The inner surface of the longitudinal body of the superior facet joint and the outer surface of the longitudinal body of the inferior facet joint may form an articulating joint, for example, like a ball and socket. U.S. patent application Ser. No. 2005/0055096 describes facet arthroplasty devices according to this description, and is incorporated herein by reference in its entirety.

In another embodiment, the facet arthroplasty device may comprise a bearing element or articulating surface intended to replace the cephalad portion of the natural facet joint, and a fixation mechanism configured to attach the artificial facet joint bearing element to the vertebra without penetrating any bone portion of the vertebra. The fixation mechanism may include a non-invasive support member configured to attach to a lamina portion of the vertebra. The facet arthroplasty device also may include an attachment mechanism attaching the artificial facet joint bearing element to the fixation mechanism. The attachment mechanism may traverse a midline of the vertebra. The attachment mechanism also may be configured such that the artificial facet joint bearing element is movable in a cephalad or caudad direction with respect to the fixation mechanism.

U.S. patent application Ser. Nos. 2004/0230304 and 2005/0010291 describe facet arthroplasty devices according to this description, and are incorporated herein by reference in its entirety.

Facet arthroplasty devices as described herein, and in general all facet arthroplasty devices, may be improved by use of a PDC on at least a portion of the articulating surfaces of the facet arthroplasty devices. In a preferred embodiment, substantially all of the at least one articulating surface of the facet arthroplasty device is a PDC. The PDC may help to improve the mechanical and physiological properties of the articulating surfaces of the facet arthroplasty devices, as has been described herein.

The PDC may be formed on at least a portion of the at least one articulating surface of the facet arthroplasty device in any applicable manner, in accordance with the guidelines provided herein. Exemplary methods by which a polycrystalline diamond compact may be formed include diamond sintering, chemical vapor deposition (CVD), physical vapor deposition (PVD), and laser ablation/deposition. One skilled in the art may recognize still other methods by which a PDC may be formed on the facet arthroplasty device, and all such methods are contemplated for use in the invention, in accordance with the guidelines provided herein.

In a preferred embodiment of the invention, a diamond sintering process may be used to form a PDC on at least a portion of the at least one articulating surface of the facet arthroplasty device. In general, the sintering process comprises sintering crystalline diamond particles to one another under high pressure and high temperature. The sintering process may be carried out on a metal substrate, for example the facet arthroplasty devices. During the sintering process, diamond-diamond, diamond-metal, and metal-metal bonds may be formed. The sintering process may result in the formation of a three-layered structure comprising the metallic substrate, a transition layer of diamond-metal, and a diamond table on the surface. The chemical and mechanical bonds between the metallic substrate and diamond table may result in very strong adhesion between the two layers.

The substrate used in the diamond sintering process may be any suitable pure metal or alloy, or a cemented carbide containing a suitable metal or alloy as a cementing agent. Preferably the substrate may be a metal with high tensile strength. Medical alloys such as titanium, titanium alloys, tantalum, tantalum alloys, stainless steel alloys, cobalt-based alloys, cobalt-chromium alloys, cobalt-chromium-molybdenum alloys, niobium alloys, and zirconium alloys also may be used in the sintering process.

The metal substrate used in the diamond sintering process may be the facet arthroplasty device itself. Alternatively, the diamond sintering process may be carried out on a metal substrate that is later attached to the facet arthroplasty device (i.e. cladding). The attachment of PDC cladding to the facet arthroplasty device can be performed using any suitable method, including, but not limited to, welding, brazing, sintering, diffusion welding, diffusion bonding, inertial welding, adhesive bonding, and the use of fasteners such as screws, bolts, or rivets.

The diamond sintering process may occur under conditions of extremely high pressure and high temperature. It has been proposed that the diamond sintering process proceeds as follows. At pressure, a cell containing feedstock of unbonded diamond powder or crystals (diamond feedstock) and a metal substrate (e.g., a facet arthroplasty device, a portion thereof, cladding, etc.) may be heated to a temperature above the melting point of the substrate metal, at which point molten metal flows or sweeps into the interstitial voids between the adjacent diamond crystals. The molten metal may be carried by the pressure gradient to fill the voids as well as being pulled in by the surface energy or capillary action of the large surface area of the diamond crystals. As the temperature continues to rise, carbon atoms from the surface of the diamond crystals may begin to dissolve into this interstitial molten metal, forming a carbon solution comprising carbon solute and metal solvent.

At the proper threshold of temperature and pressure, diamond becomes the thermodynamically favored crystalline allotrope of carbon. As the solution becomes super saturated with respect to carbon diamond, carbon from this solution may begin to crystallize as diamond onto the surfaces of the diamond crystals, thereby bonding adjacent diamond crystals together with diamond-diamond bonds into a sintered polycrystalline diamond structure. The interstitial metal may fill the remaining void space, forming a vein-like lattice structure within the diamond table by capillary forces and pressure driving forces. Because of the important role that the interstitial metal plays in forming a solution of carbon atoms and stabilizing these reactive atoms during the diamond crystallization phase, the metal may be referred to as a solvent-catalyst metal.

The diamond sintering process may result in the formation of a PDC with a three-tiered structure comprising a metal substrate, a diamond table, and a transition zone between the diamond table and the metal substrate. The transition zone also can be called an interface, a gradient transition zone, a composition gradient zone, or a composition gradient. The transition zone represents a gradient interface between the diamond table and the substrate with a gradual transition of ratios between diamond content and metal content. At the substrate side of the transition zone, there may be only a small percentage of diamond crystals and a high percentage of substrate metal, and on the diamond table side, there may be a high percentage of diamond crystals and a low percentage of substrate metal.

In the transition zone where diamond crystals and substrate metal are intermingled, chemical bonds may be formed between the diamond and metal. From the transition zone into the diamond table, the metal content may diminish and may be limited to metal that fills the three-dimensional vein-like structure of interstitial voids or openings within the sintered diamond table structure. The transition zone may distribute diamond/substrate stress over the thickness of the zone, thereby reducing the residual stresses that are created due to the difference in pressure and thermal expansive properties of the diamond and substrate materials as pressure and temperature are reduced at the conclusion of the high pressure, high temperature sintering process.

During the sintering process, there may be three types of chemical bonds created: diamond-to-diamond bonds, diamond-to-metal bonds, and metal-to-metal bonds. In the diamond table, there may be diamond-to-diamond bonds (sp3 carbon bonds) created when diamond particles partially solvate in the solvent-catalyst metal and then are bonded together. In the substrate and in the diamond table, there may be metal-to-metal bonds created by the high pressure and high temperature sintering process. And in the gradient transition zone, diamond-to-metal bonds may be created between diamond and solvent-catalyst metal.

The combination of the various chemical bonds and the mechanical grip exerted by solvent-catalyst metal in the diamond table (e.g., in the interstitial spaces of the diamond table) may provide extraordinarily high bond strength between the diamond table and the substrate. This bonding structure may contribute to the extraordinary fracture toughness of the compact, and the veins of metal within the diamond table may act as energy sinks halting propagation of incipient cracks within the diamond structure. The transition zone and metal vein structure may provide the compact with a gradient of material properties between those of the diamond table and those of substrate material, further contributing to the extreme toughness of the compact.

In other embodiments of the invention, a boundary layer of a third material different than the diamond and the substrate may be placed at the interface. This interface boundary layer material may serve several functions including, but not limited to, enhancing the bond of the diamond table to the substrate, and mitigation of the residual stress field at the diamond-substrate interface. However, the interface layer may prevent metal from the substrate from being swept into the diamond table to participate in the sintering process. In this case, the boundary layer material, if composed of a suitable material, metal, or alloy that can function as a solvent-catalyst, itself may serve as the sweep material mediating the diamond sintering process.

In other cases, for example where the desired boundary material cannot serve as a solvent-catalyst, a suitable amount of solvent-catalyst metal powder may be added to the diamond crystal feedstock. The metal may be added by direct addition of powder, or by generation of metal powder in situ using an attritor mill, by the well-known method of chemical reduction of metal salts deposited on diamond crystals, vapor deposition, or any other applicable method, following the guidelines herein. Added metal may constitute any amount from less than 1% by mass, to greater than 35% by mass, of the diamond feedstock/solvent-catalyst metal mixture. This added metal may consist of the same metal or alloy as is found in the substrate, or may be a different metal or alloy selected, for example, because of its material and mechanical properties.

Besides instances where a boundary layer that cannot function as a solvent-catalyst is placed at the interface, inclusion of solvent-catalyst metal in the diamond feedstock also may be useful in any other situation where there is insufficient ingress of solvent-catalyst metal via the sweep mechanism to adequately mediate the sintering process as a solvent-catalyst. For example, it may be desirable to add solvent-catalyst metal to the diamond feedstock when forming thick PDC tables, solid PDC structures, or when using multimodal fine diamond where there is little residual free space within the diamond powder.

Another modification of the diamond sintering process comprises the fabrication of a PDC structure separate from the facet arthroplasty device itself. The PDC structure may function as at least a portion of an articulating surface of a facet arthroplasty device. This may be done by placing the diamond powder combined with a suitable amount of added solvent-catalyst metal powder as described above in a refractory metal can (e.g., tantalum, niobium, zirconium, or molybdenum) with a shape approximating the shape of the final part desired. This assembly then may be taken through the sintering process as described herein. However, in the absence of a substrate metal source, the solvent-catalyst metal for the diamond sintering process must be supplied entirely from the added metal powder. With suitable finishing, objects thus formed may be used as is, or bonded to metal substrates, to function as at least a portion of a facet arthroplasty device.

In another embodiment of the invention, chemical vapor deposition (“CVD”) and physical vapor deposition (“PVD”) processes may be used to form a polycrystalline diamond compact on at least a portion of the at least one articulating surface of the facet arthroplasty device. CVD and PVD processes deposit a layer of polycrystalline diamond, optionally mixed with other materials, on a substrate material such as titanium, a carbide, silicon, or molybdenum. Application of a polycrystalline layer to a substrate forms a composite comprising the substrate and the polycrystalline layer; the composite is a “polycrystalline diamond compact,” as the phrase is used herein. Therefore, coating of at least a portion of a facet arthroplasty device with polycrystalline diamond may produce a PDC on the coated portion of the facet arthroplasty device.

CVD generally takes place in a reactor with one or more gas inlets and exit ports, a stage on which to place the substrate (e.g. the facet arthroplasty device on which the PDC is to be formed), and a thermal source to heat the gases in the chamber and/or the facet arthroplasty device. The general process by which a CVD process proceeds is as follows.

A substrate, for example a facet arthroplasty device, may be placed into the reactor chamber. Reactants may be introduced to the chamber via one or more gas inlets. For diamond CVD, methane (CH4) and hydrogen (H₂) gases preferably are brought into the chamber in a premixed form. Alternatively, instead of methane, any carbon-bearing gas in which the carbon has sp3 bonding also may be used. Other gases such as oxygen, carbon dioxide, argon, and halogens may be added to the gas stream in order to control the quality of the diamond film, deposition temperature, grain structure and growth rate.

In a preferred embodiment, the gas pressure in the chamber may be maintained at about 100 torr. Flow rates for the gases through the chamber preferably may be about 10 standard cubic centimeters per minute for methane and about 100 standard cubic centimeters per minute for hydrogen. The composition of the gas phase in the chamber preferably may be in the range of 90-99.5% hydrogen and 0.5-10% methane.

The gases may be heated as they are introduced into the chamber. Heating may be accomplished by any applicable method. In a plasma-assisted process, the gases are heated by passing them through a plasma. Otherwise, the gases may be passed over a series of wires such as those found in a typical hot filament reactor. Heating the methane and hydrogen may cause them to break down into various free radicals. Through a complicated mixture of reactions, carbon may be deposited on the substrate and join with other carbon atoms to form polycrystalline diamond by sp3 bonding. The atomic hydrogen in the chamber may react with and remove hydrogen atoms from methyl radicals attached to the substrate surface, leaving a clear solid surface for further deposition of free radicals and continued growth of the polycrystalline diamond layer.

The gas composition, flow rates, substrate temperature, and other variables may need to be adjusted within certain parameters in order to affect the formation of polycrystalline diamond on the substrate rather than graphite. One who is skilled in the art will be able to choose appropriate reaction conditions to ensure the formation of polycrystalline diamond, in accordance with the limitations described herein.

In another embodiment of the invention, physical vapor deposition (“PVD”) may be used to form a PDC on at least a portion of the at least one articulating surface of the facet arthroplasty device. PVD generally takes place in a reactor with one or more gas inlets and exit ports, a stage on which to place the substrate (e.g. the facet arthroplasty device on which a PDC is to be formed), and a source of plate material. The general process by which the PVD process proceeds is as follows.

A PVD reactor generates electrical bias across a plate of source material in order to generate and eject carbon radicals from the source material. For example, the reactor may bombard the source material with high energy ions. When the high energy ions collide with the source material, they may cause ejection of the desired carbon radicals from the source material. The ejected carbon radicals then may deposit themselves onto whatever is in their path, including the stage, the reactor itself, and the substrate (e.g., the facet arthroplasty device).

Because both CVD and PVD processes achieve polycrystalline diamond deposition by line-of-sight, methods such as vibration and rotation of the stage may be utilized to expose all desired surfaces for polycrystalline diamond deposition. For example, a vibratory stage may be used wherein the surface that is to be coated vibrates up and down with the stage and thereby presents more or all of the surface to the free radical source.

It may be desirable that only a portion of a facet arthroplasty device be coated with polycrystalline diamond using a CVD or PVC process in order to form a PDC. In a preferred embodiment, only the at least one articulating surface of the facet arthroplasty device has a PDC formed thereon. Therefore, the portions of the facet arthroplasty device where a PDC are not intended to be formed may be covered, for example with a mask or other coating, in order to prevent PDC formation on those portions of the device. Following PDC formation, the mask may be removed to reveal the underlying substrate. In this way, a PDC may be selectively formed on portions of the facet arthroplasty device using CVD and PVD processes.

Alternatively, like diamond sintering, CVD and PVD processes may be used to apply a polycrystalline diamond coating to a substrate (i.e. cladding) to form a PDC that is subsequently attached to the facet arthroplasty device. The polycrystalline diamond coated substrate can be referred to as a PDC because it is a composite of polycrystalline diamond and at least one other material (i.e., the substrate or cladding). The cladding then may be attached to the facet arthroplasty device using any suitable attachment method, including welding, brazing, sintering, diffusion welding, diffusion bonding, inertial welding, adhesive bonding, and the use of fasteners such as screws, bolts, or rivets.

Additionally, CVD and PVD processes may be utilized in order to produce free standing polycrystalline diamond structures that are later attached to the facet arthroplasty device by welding, diffusion bonding, adhesion bonding, mechanical fixation, high pressure and high temperature, or by another applicable method. Again, because the final structure is a composite comprising the facet arthroplasty device as a substrate and the polycrystalline diamond coating on the substrate, the polycrystalline diamond coated surfaces may be referred to as a PDC.

In another alternative, CVD and PVD processes can be conducted in a manner such that metal is incorporated into the polycrystalline diamond table. Incorporation of metal into the polycrystalline diamond table may enhance adhesion of the polycrystalline diamond table to its substrate and can strengthen the resulting polycrystalline diamond compact. Incorporation of metal into the polycrystalline diamond table also can be used to achieve a polycrystalline diamond and metal table with a coefficient of thermal expansion and compressibility different from that of pure polycrystalline diamond, resulting in increased fracture toughness of the polycrystalline diamond and metal table as compared to pure polycrystalline diamond. Diamond has a low coefficient of thermal expansion and a low compressibility compared to metals. Therefore the presence of metal with diamond in the polycrystalline diamond and metal table may achieve a higher and more metal-like coefficient of thermal expansion and average compressibility for the polycrystalline diamond and metal table than for pure polycrystalline diamond. Consequently, residual stresses at the interface of the polycrystalline diamond and metal table and the substrate may be reduced, and delamination of the polycrystalline diamond and metal table from the substrate is less likely.

CVD and PVD processes also can be conducted so that a transition zone is established. Like the transition zone that may be created by diamond sintering, the transition zone created by modified CVD and PVD process may represent a transition gradient between the predominantly metal substrate and the diamond table on the surface. It is preferred that the outer most surface be essentially pure polycrystalline diamond in order to attain the best wear properties.

One method for incorporating metal into a CVD or PVD diamond film is to use two different source materials in order to simultaneously deposit the two materials on a substrate during a CVD or PVD diamond production process. This method may be used regardless of whether polycrystalline diamond is being produced by CVD, PVD, or a combination of the two.

Another method for incorporating metal into a CVD polycrystalline diamond film is chemical vapor infiltration. According to this process, a porous layer may first be created on the substrate's surface (e.g., the surface of the facet arthroplasty device), and then the pores may be filled with a metal by a chemical vapor infiltration process. The porous layer thickness should be approximately equal to the desired thickness for the transition or gradient layer. The size and distribution of the pores can be used to control the ultimate composition of the layer. Deposition via vapor infiltration may occur first at the interface between the porous layer and the substrate. As deposition continues, the interface along which the material is deposited may move outward from the substrate to fill the pores in the porous layer. As the growth interface moves outward, the deposition temperature along the interface may be maintained by moving the sample relative to a heater or by moving the heater relative to the growth interface. It is desirable that the porous region between the outside of the sample and the growth interface be maintained at a temperature that does not promote deposition of material (either the pore-filling material or undesired reaction products) because deposition in this region can close the pores prematurely and prevent infiltration and deposition of the desired material in deeper pores. The result would be a substrate with open porosity and poor physical properties.

In another embodiment of the invention, energy beam ablation/deposition may be used to form a PDC layer on at least a portion of the surface of a facet arthroplasty device. Energy beam ablation/deposition processes use an energy beam such as laser energy to vaporize constituents in a substrate and redeposit those constituents on the substrate in a new form, such as in the form of a polycrystalline diamond coating. For example, a metal, polymeric, or other substrate may be obtained or produced containing carbon, carbides or other desired constituent elements. Appropriate energy, such as laser energy, may be directed at the substrate to cause constituent elements such as carbon to move from within the substrate to its surface in areas adjacent to the area of application of energy to the substrate. Continued application of energy to the concentrated constituent elements on the surface of the substrate can be used to cause vaporization of some of those constituent elements. The vaporized constituents then may be reacted with other elements to change the properties and structure of the vaporized constituent elements. For example, vaporized carbon may react with other carbon to form diamond. Next, the vaporized and reacted constituent elements (e.g., diamond) may be diffused into the surface of the substrate. By this process and variations of it, coatings of diamond may be formed on a substrate. The final composite structure of diamond and substrate is a PDC.

Besides diamond sintering, CVD, PVD, and energy beam ablation/deposition processes, the invention contemplates that other procedures may be utilized to form a PDC on at least a portion of an articulating surface of a facet arthroplasty device. One skilled in the art will appreciate other methods by which this is to be accomplished, in accordance with the guidelines discussed herein.

Following formation of a PDC on at least a portion of the articulating surface of the facet arthroplasty device, the PDC may be polished smooth in order to attain an exceptionally low coefficient of friction. Polishing may proceed in any applicable manner, as will be appreciated by one of skill in the art. Preferrably, polishing may also be used in order to remove PDC material and attain the desired final dimensions of the PDC articulating surface of the facet arthroplasty device.

As has been described, polycrystalline diamond compacts (PDC) are useful surfaces for facet arthroplasty devices, particularly for articulating surfaces of facet arthroplasty devices. The polycrystalline diamond compacts contemplated by the invention can be formed by any applicable fashion to any applicable facet arthroplasty device, in accordance with the guidelines discussed herein.

In a preferred embodiment, the polycrystalline diamond compact may be polished or highly polished. This may be advantageous in order to decrease the coefficient of friction of the PDC articulating surface. The articulating surfaces having PDC portions, as provided by embodiments of the invention, may be useful articulating surfaces for mating with a number of other surfaces against which it articulates. For example, the articulating surface may articulate against a ceramic such as alumina, zirconia, and pyrolytic carbon; a metallic alloy such as cobalt alloy and titanium alloy; a polymer such as ultra-high molecular weight polyethylene, polyetheretherketone, and polyurethane; and a natural tissue such as cartilage, bone, and soft tissues.

The foregoing detailed description is provided to describe the invention in detail, and is not intended to limit the invention. Those skilled in the art will appreciate that various modifications may be made to the invention without departing significantly from the spirit and scope thereof.

What is claimed is:

1. A facet arthroplasty device comprising at least one articulating surface, wherein at least a portion of the at least one articulating surface is a polycrystalline diamond compact.

2. The facet arthroplasty device of claim 1, wherein substantially all of the at least one articulating surface is a polycrystalline diamond compact.

3. The facet arthroplasty device of claim 1, wherein the Knoop hardness of the articulating surface is greater than about 6000.

4. The facet arthroplasty device of claim 1, wherein the polycrystalline diamond compact is formed by a diamond sintering process.

5. The facet arthroplasty device of claim 1, wherein the polycrystalline diamond compact is formed by a chemical vapor deposition process.

6. The facet arthroplasty device of claim 1, wherein the polycrystalline diamond compact is formed by a physical vapor deposition process.

7. The facet arthroplasty device of claim 1, wherein the polycrystalline diamond compact is formed by an energy beam ablation/deposition process.

8. The facet arthroplasty device of claim 1, wherein the polycrystalline diamond compact is fabricated separately from the facet arthroplasty device and then is attached to the facet arthroplasty device.

9. The facet arthroplasty device of claim 8, wherein the polycrystalline diamond compact is attached to the facet arthroplasty device by a method selected from the group consisting of: welding, brazing, sintering, diffusion welding, diffusion bonding, inertial welding, adhesive bonding, and the use of fasteners including screws, bolts, and rivets.

10. The facet arthroplasty device of claim 1, wherein the polycrystalline diamond compact comprises a metallic substrate, a layer of polycrystalline diamond, and a transition layer positioned between the metallic substrate and the polycrystalline diamond.

11. The facet arthroplasty device of claim 1, wherein the polycrystalline diamond compact is polished or highly polished.

12. The facet arthroplasty device of claim 1, wherein the articulating surface articulates against another polycrystalline diamond compact.

13. The facet arthroplasty device of claim 1, wherein the articulating surface articulates against a ceramic selected from the group consisting of alumina, zirconia, and pyrolytic carbon.

14. The facet arthroplasty device of claim 1, wherein the articulating surface having a polycrystalline diamond compact articulates against a metallic alloy selected from the group consisting of cobalt alloy and titanium alloy.

15. The facet arthroplasty device of claim 1, wherein the articulating surface having a polycrystalline diamond compact articulates against a polymer selected from the group consisting of ultra-high molecular weight polyethylene, polyetheretherketone, and polyurethane.

16. The facet arthroplasty device of claim 1, wherein the articulating surface having a polycrystalline diamond compact articulates against natural tissues selected from the group consisting of cartilage, bone, and soft tissues.

17. A method for modifying a facet arthroplasty device having at least one articulating surface, comprising forming a polycrystalline diamond compact on at least a portion of the at least one articulating surface.

18. The method of claim 17, further comprising polishing the polycrystalline diamond compact following forming the compact on at least a portion of the at least one articulating surface.

19. The method of claim 17, wherein the polycrystalline diamond compact covers substantially all of the articulating surfaces of the facet arthroplasty device.

20. The method of claim 17, wherein the articulating surfaces of the facet arthroplasty device that are covered with a polycrystalline diamond compact have a Knoop hardness of greater than about 6000.

21. The method of claim 17, wherein forming a polycrystalline diamond compact comprises performing a diamond sintering process.

22. The method of claim 17, wherein forming a polycrystalline diamond compact comprises performing an energy beam ablation/deposition process.

23. The method of claim 17, wherein forming a polycrystalline diamond compact comprises performing a chemical vapor deposition process.

24. The method of claim 17, wherein forming a polycrystalline diamond compact comprises performing a physical vapor deposition process.

25. The method of claim 17, further comprising polishing or highly polishing the polycrystalline diamond compact.
